(19) United States
(12) Patent Application Publication
(10) Pub. No.: US 2021/0162089 A1
(43) Pub. Date: Jun. 3, 2021

(54) ULTRAVIOLET FACE MASK

(71) Applicants: Thomas Wolfgang Zimmermann, San Jose, CA (US); Leonardo Wolfgang Machado, San Jose, CA (US)

(72) Inventors: Thomas Wolfgang Zimmermann, San Jose, CA (US); Leonardo Wolfgang Machado, San Jose, CA (US)

(21) Appl. No.: 17/170,198

(22) Filed: Feb. 8, 2021

Related U.S. Application Data
(60) Provisional application No. 62/995,744, filed on Feb. 10, 2020.

(51) Int. Cl. A61L 9/20; A41D 13/11; A62B 23/02; A62B 9/02; A62B 9/00

(52) U.S. Cl. CPC A61L 9/20; A41D 13/1192; A41D 13/1138; A61L 2209/14; A62B 9/02; A62B 9/006; A61L 2209/12; A62B 23/025

(57) ABSTRACT

A face mask assembly that includes a cover, a V-shape shell, a battery, a UV-C LED chipset having one or more UV-C LEDs, and a mouthpiece. The cover having apertures for ingress and egress of air between an inner volume of the V-shape shell and the outside environment. The V-shape shell can fit into the cover forming the inner volume between two walls of the V-shape shell and the cover. In one wall of the V-shape shell is a window that is having a UV-C LED for irradiating the inner volume of the V-shape shell. The other wall of the V-shape shell having apertures for inhaling from the inner volume and exhaling into the inner volume. The face mask can sterilize both the inhaled air and the exhaled air using the pulsating UV-C LEDs.

### CROSS-REFERENCE TO RELATED APPLICATIONS

This application claims priority to the U.S. provisional patent application Ser. No. 62/995,744, filed on Feb. 10, 2020, which is incorporated herein by reference in its entirety.

### FIELD OF INVENTION

The application relates to a face mask assembly, and more particularly, this application relates to an ultraviolet face mask assembly.

### BACKGROUND

Face masks covering the nose and mouth are widely used to protect from airborne contaminants and microorganisms entering the respiratory system. A face mask acts as a shield catching the particles and microbes before they could enter the respiratory system. The basic principle behind the face masks is filtration, wherein the face mask retains the particles larger than its pore size. However, to be effective against virions and similar microorganisms, face masks with multiple layers are generally used. Virions are minute in size and could pass the fabric layer of the filter. Multilayer face masks, such as N95 face masks have at least one electrocharged layer that attracts virions and bacteria.

Although face masks are available that can trap microorganism including virions, however, known face mask has several drawbacks which limit their effectiveness. First, the captured microorganisms get accumulated over a layer of the face mask decreasing its performance with time. The short life of face masks increases the overall cost of protection. The presence of a large number of microorganisms on the face mask can itself become a source of contamination. The fabric or electrostatic layer of face masks has limited microbe trapping efficiency and some microbes, specifically, virions can make their way through the face mask into the respiratory system.

Thus, a long-standing need is there for a face mask that is devoid of the above drawbacks of the known face masks.

### SUMMARY OF THE INVENTION

The principal object of the present invention is therefore directed to a face mask assembly that reduces the microbial load accumulated on the filter of the face mask.

It is another object of the present invention that the face mask assembly provides additional safety by directly killing the microorganisms.

It is still another object of the present invention that the face mask assembly can be used for longer durations.

It is still a further object of the present invention that the face mask assembly can be customized by replacing the filter or the mouthpiece.

It is yet another object of the present invention that the face mask assembly is economical to use.

It is still another object of the present invention that the face mask assembly can be used without the filter.

It is still an additional object of the present invention that the facemask assembly provides for sterilization of both inhaled and exhaled air.

In one aspect, disclosed is a face mask assembly having a cover, UVC LED chipset, V-shaped shell, a mouthpiece, and a battery. The UVC LED chipset, V-shaped shell, mouthpiece, and the battery are all enclosed in the cover. The mouthpiece is of an ergonomic design that can fit perfectly on a face around the nose and mouth of a wearer. The cover can have inhalation apertures and exhalation apertures that open into the interior of the V-shaped shell. The V-shaped shell having breathing apertures for both the inhalation of air from the interior of the V-shaped shell and exhalation of air into the interior of the V-shaped shell.

In one aspect, the V-shaped shell having a window on a wall that is opposite the wall of the V-shaped shell having the apertures of inhalation and exhalation of air. One or more UV-LED can be mounted in the window of the V-shaped shell to irradiate the inner volume of the V-shaped shell. The inner surface of the V-shaped shell can have a coating of reflective material to enhance the efficiency of the UV-C irradiations.

In one aspect, disclosed is a face mask assembly that includes a housing having a front face, a rear face, a left face, a right face, a top face, and a bottom face defining an inner volume of the housing. The front face of the housing having a first aperture for air intake. A bracket is configured around the aperture on the outer side of the front face. The bracket is three-sided having a continuous slot that can slidably receive a filter, wherein the filter acts as a barrier between the outer atmosphere and inner volume of the housing. The filter can be in a form of a wafer that can be replaced by pulling out the old one from the bracket and replacing it with a new one. The filter can be an N95 filter or a similar filter known to a skilled person for use in face masks. The rear face of the housing includes a second aperture for air egress from the housing. A joint is configured around the second aperture, wherein the joint can couple with a nose and mouth facepiece. The nose and mouth facepiece can cover the nose and mouth and includes straps to secure the disclosed face mask assembly to the face.

In one aspect, the housing includes a PCB having at least one UV LED that irradiates the inner volume of the housing and the filter attached to the first aperture. The UV LED can be powered by a portable and rechargeable battery also enclosed in the housing. The second aperture having the air exit can be protected by a baffle system that shields the ultraviolet radiations but allows the ingress and egress of air through the baffles.

These and other objects and advantages of the embodiments herein will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a perspective view of a face mask assembly shown to be worn over a face, according to an exemplary embodiment of the present invention.

FIG. 2 is a front view of the face mask assembly having a transparent cover to show the inner components, according to an exemplary embodiment of the present invention.

FIG. 3 is a sectional side-view of the face mask assembly, according to an exemplary embodiment of the present invention.

FIG. 4 is an exploded view of the face mask assembly, according to an exemplary embodiment of the present invention.

FIG. 5 is a perspective view of another embodiment of the face mask assembly showing the housing, bracket, filter wafer, and facepiece, according to the present invention.

FIG. 6 is the rear face of the housing of the face mask assembly shown in FIG. 5, according to an exemplary embodiment of the present invention.

FIG. 7 is a right-side view of the housing of FIG. 6, according to an exemplary embodiment of the present invention.

FIG. 8 is a left side view of the housing of FIG. 6, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

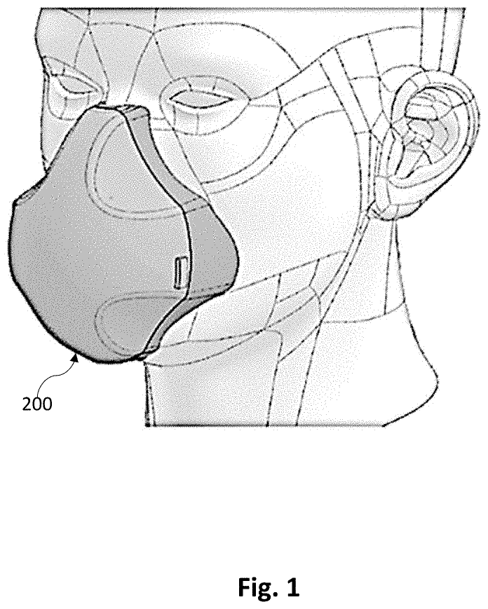

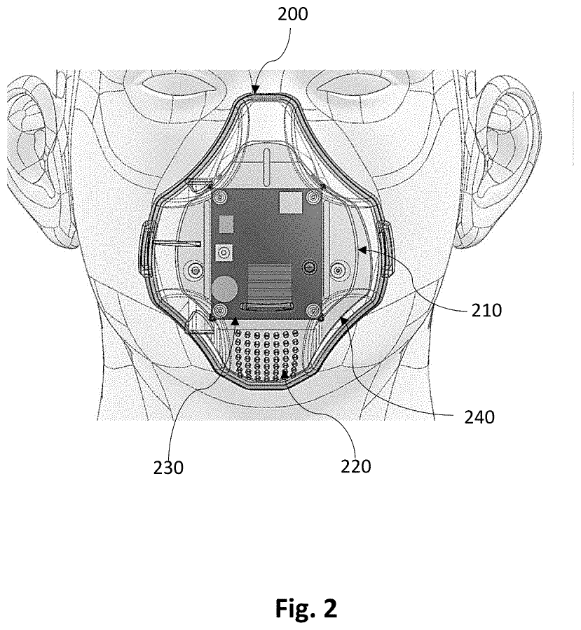

Referring to FIG. 1, which is a perspective view of the disclosed face mask assembly 200 shown to be worn over a face of a person. FIG. 2 is the front view of the face mask assembly 200, however, the cover of the face mask assembly is transparent for illustrating the inner components. It can be seen in FIG. 2, the disclose face mask assembly 200 can include a cover 210, the cover 210 having apertures 220, a UV-C LED chipset 230, and a V-shaped Shell 240. The cover also acts as housing that can enclose the above components of the face mask assembly 200. The cover can be made of any lightweight material, such as plastic or lightweight metal, such as Aluminum.

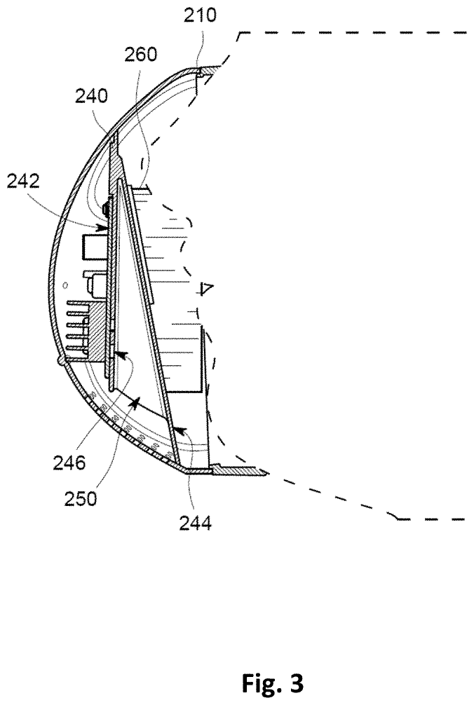

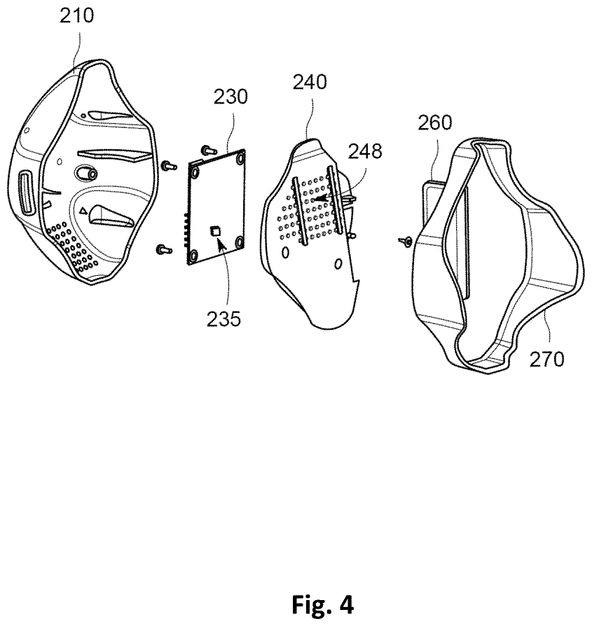

FIG. 2 shows apertures 220 in the lower area of the cover 210. The apertures also more clearly shown in FIG. 3 and FIG. 4 opens in the inner volume 250 of the V-shaped shell 240. The apertures 220 can be of two types i.e., the inhalation apertures and exhalation apertures. The inhalation apertures can be interrupted by a one-way silicone flapper valve that can permit the air from outside to ingress into the inner volume of the V-shaped shell 240. The exhalation apertures can also be interrupted by a one-way silicone flapper valve that can permit the air from the inner volume of the V-shaped shell to egress outside the face mask assembly 200. The one-way silicone flapper valve can be a silicon wafer with a split that allows air under pressure to flow in one direction only.

FIG. 3 shows a sectional side view of the face mask 200 showing the apertures 220 open into the inner volume of the V-shaped shell 240. The V-shape shell 240, also shown in the exploded view in FIG. 4, is having a front wall 242 and a rear wall 244, such as the two walls extends at an acute angle from top to bottom. The V-shape shell 240 can fit into the cover 110, such as to form an enclosed interior volume 250 between the V-shape shell 240 and the inner surface of the cover 210. The front wall 242 of the V-shape shell 240 is having a window 246 into which a UV-C LED can be installed. UV-C LED can be configured in a chipset 230 that can have the circuitry for generating pulses on a millisecond basis. In one case, two UV-C LEDs can be provided, wherein the first UV-C LED can be made to pulse at 32 milliseconds On and 68 milliseconds Off. The second UV-C LED can be pulsing 25 milliseconds On and 75 milliseconds Off. FIG. 4 shows the UV-C LED 235 on the UV-C LED chipset 230. Again, referring to FIG. 3, the window 246 in the front wall 242 of the V-shape shell 240 is in the lower part of the wall which can irradiate the inner volume 250, and particularly, the inner volume adjacent to the apertures 220. For this, the UV-C LEDs can be angled downward at a 15° angle towards the apertures 220. FIG. 4 shows the rear wall 244 of the V-shape shell 240 having apertures in the upper portion for exchange the air between the inner volume 250 of the V-shape shell 240 and the space between the rear wall of the V-shape shell 240 and the face of a person wearing the disclosed assembly. As the user inhales or exhales, the air can be exchanged through the apertures 248 on the rear wall of the V-shape shell 240.

The UV-C LED chipset can be powered by the battery 260 that can be mounted inside cover 210, as shown in FIGS. 2 and 3. A mouthpiece 270 is coupled around the periphery of the cover 210, wherein the mouthpiece 270 is of a shape that is commensurate with the contours around the nose and mouth of a person. The mouthpiece 270 allows a close-fitting of the disclosed mask assembly over a face of a wearer.

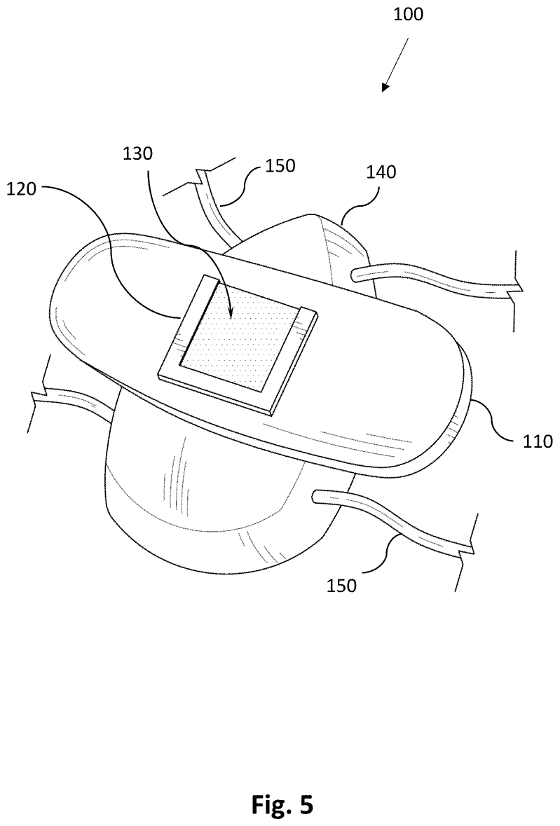

Referring to FIG. 5 which illustrates another exemplary embodiment of the disclosed face mask assembly. The disclosed face mask assembly provides additional safety by killing the microorganism accumulated on the filter and the microorganism that passes through the filter. FIG. 5 shows the face mask assembly 100 having a central housing 110 that includes a front face, a rear face, a left face, a right face, a top face, and a bottom face defining an inner volume of the housing. The housing 110 shown in FIG. 5 is a cuboid shape having rounded edges. Moreover, the left side and the right side of the housing can also be curved. However, the housing could be made of any other shape without departing from the scope of the present invention. Considering the shape of the face, a wider and thinner housing, as shown in FIG. 5, is preferable. In one exemplary embodiment, the housing can be made of a rigid, durable, and ultraviolet resistant material. For example, the housing can be injection molded from UV stabilized high-density polyethylene or PVC.

The front face of the housing 110 can have a wide aperture for air intake from the outer atmosphere. Also, referred to herein as an inlet port, the wide aperture can be of a dimension ranging from round to square or rectangular. A bracket 120 in the form of three-sided slots can be seen in FIG. 5 configured around the inlet port and on the outer side of the front face. The bracket includes a continuous channel running through the three slots. One side of the bracket is open and can receive a wafer shaped filter 130. The filter wafer 130 can be inserted into the channel and secured by a locking mechanism. For example, the filter wafer can be snap-fit into the bracket. The filter wafer can be snugly received into the bracket such as to form an airtight seal around the inlet port. It is to be understood that FIG. 5 shows a three-sided bracket, however, any other mechanism for mounting a filter to the housing is within the scope of the present invention. For example, a frame can be configured around the inlet port on which the filter wafer can be placed, and a flange secures the filter wafer to the frame. The filter wafer can be removable from the bracket allowing the filter to be replaced by a new one or a different filter. In one case, the locking mechanism of the filter can be unlocked, and the filter wafer pulled out from the bracket. The locking and unlocking mechanism can be optional and the filter wafer can also be frictionally retained. All types of air filters known for use in face masks are within the scope of the present invention. For example, the filter can be an N95 filter.

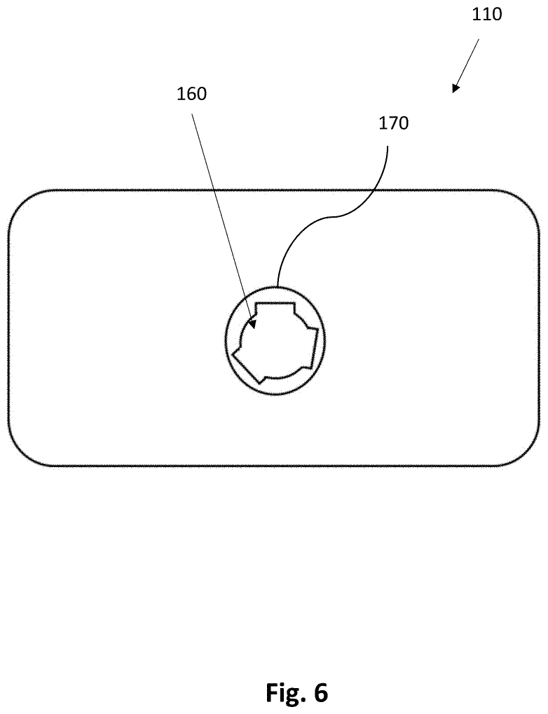

Referring to FIG. 6 showing the rear face of the housing 110. The rear face of the housing includes a second aperture 160, also referred herein as the outlet of the housing. The second aperture is an outlet for the air from the inner volume of the housing. The air from the inner volume of the housing can be inspired through the second aperture and the air from breathing can be exhaled into the inner volume through the second aperture. Filtered air can be received into the housing from the first aperture, and sterilized air can exit from the housing through the second aperture. Exhaled air from breathing can also be received through the second aperture into the inner volume and sterilized. A joint 170 is configured around the second aperture 160 on the outer side of the rear face. The joint can be used to couple with a nose and mouth facepiece 140 also referred herein as the facepiece or mouthpiece. The facepiece 140 covers the nose and mouth of a face. The structure and functioning of the facepiece are known to a skilled person and such known facepieces are within the scope of the present invention. The facepiece includes a part of the joint, such as the facepiece can be removably coupled to the joint on the rear face of the housing. In one case, the housing has a first member of a joint and the facepiece has a second member of the joint. The first member and the second member can be two parts of a snap-fit joint or a slide and lock type of joint. The facepiece can include straps 150 for securing the face mask assembly to the face. The face mask assembly shown in FIG. 5 includes a head strap and a neck strap.

The second aperture can also be protected by a baffle for shielding the face from UV radiations. The baffle can be a series of partially overlapping plates covering the second aperture. The space between overlapping portions of the plate may provide a passage of air to flow in either direction i.e., air can egress from the inner volume of the housing through the baffle's spacings and exhaled air can ingress into the inner volume through the baffle's spacings.

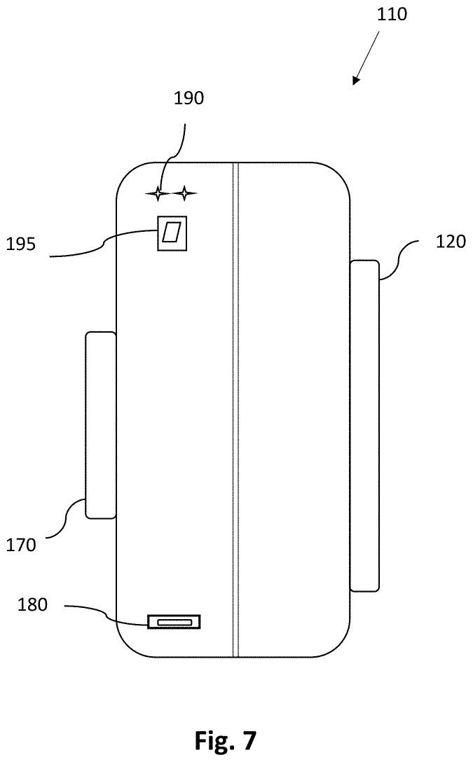

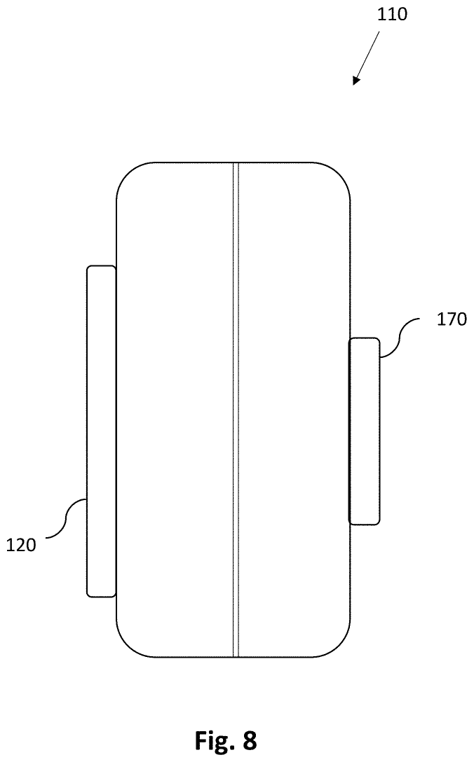

Referring to FIGS. 7 and 8 showing the left side and the right side of the housing. The housing includes a PCB that includes at least two UVC LEDs for sterilizing the air contained in the inner volume of the housing. The inner volume of the housing can contain the filtered air and exhaled air from breathing. The filter can also be irradiated from inside killing the accumulated microbes on the filter. The PCB causes the two UVC LEDs to pulse on a millisecond basis. The first UVC LED can be made to pulse at 32 milliseconds on and 68 milliseconds off. The second UVC LED can be pulsing 25 milliseconds on and 75 milliseconds off. The combination of the two pulsating UVC LEDs was found to be effective in sterilizing the filtered air and exhaled air in a very short time.

The PCB can be powered by a battery also encased in the housing. In one case, the battery can be a rechargeable battery, for example, a lithium-ion battery. A charging port 180 connected to the charging circuitry can be provided on the housing 110. The charging circuitry can charge the battery. The charge level of the battery can also be indicated. In one case, a visual indicator 190 can be provided on the housing that has a red color indicating low battery, a yellow color indicating medium charge level, and green color indicating that the battery is charged almost full. The visual indicator can be provided adjacent to the charging port or anywhere in the housing. FIG. 7 also shows a power button 195 for turning the power supply to the PCB on and off.

What is claimed is:

1. A face mask assembly comprising:
a V-shape shell having a front wall and a rear wall, the front wall and the rear wall having a common top edge and extends downwards at an acute angle;
one or more windows in the front wall;
a plurality of breathing apertures in an upper portion of the rear wall;
a cover having a profile of a face mask that can cover a nose and a mouth of a wearer, the V-shape shell fit into the cover forming an enclosed inner volume between the front wall, the rear wall, and an inner surface of the cover;
a plurality of ingress apertures and a plurality of egress apertures in the cover that open in the inner volume; and
one or more UV-C LEDs mounted into the one or more windows for irradiating the inner volume.

2. The face mask assembly according to claim 1, wherein the plurality of ingress apertures configured to permit ingress of air from outside into the inner volume, the plurality of egress apertures configured to permit egress of air from the inner volume to the outside.

3. The face mask assembly according to claim 1, wherein the one or more UV-C LEDs are positioned at a downward angle towards the plurality of ingress apertures and the plurality of egress apertures.

4. The face mask assembly according to claim 1, wherein the rear wall is positioned vertical, and the front wall is slanted relative to the cover.

5. The face mask assembly according to claim 4, wherein the breathing apertures are positioned in the rear wall such as the breathing apertures are in front of the nose and the mouth when face mask assembly is worn.

6. The face mask assembly according to claim 1, wherein the one or more UV-C LEDs are installed in a PCB board coupled to the front wall of the V-shape shell.

7. The face mask assembly according to claim 6, wherein two UV-C LEDs are installed in the PCB board.

8. The face mask assembly according to claim 7, wherein the PCB board is configured to cause the two UV LEDs to pulse on a millisecond basis, a first UV LED of the two UV LEDs made to pulse at 32 milliseconds on and 68 milliseconds off and a second UV LED of the two UV LEDs made to pulse at 25 milliseconds on and 75 milliseconds off.

9. The face mask assembly according to claim 8, wherein the face mask assembly further comprises a battery enclosed in the cover, the battery configured to power the PCB board.
